(19) United States
(12) Patent Application Publication
(10) Pub. No.: US 2003/0135222 A1
(43) Pub. Date: Jul. 17, 2003

(54) Surgical snare

(76) Inventor: Kanag Baska, Strathfield (AU)

Correspondence Address: SCHNADER HARRISON SEGAL & LEWIS, LLP, 1600 MARKET STREET, SUITE 3600, PHILADELPHIA, PA, 19103

(21) Appl. No.: 10/204,146

(22) PCT Filed: Feb. 16, 2001
(86) PCT No.: PCT/AU01/00159

(30) Foreign Application Priority Data
Feb. 17, 2000 (AU) PQ 5685
Jun. 16, 2000 (AU) PQ 8182

(51) Int. Cl. A61B 17/24

(52) U.S. Cl. 606/113

(57) ABSTRACT

A surgical snare (10) includes an elongate barrel (11) having a distal end (12) and a proximal end (13). A handle (14) having holes (15, 16) which can accommodate the fingers of a surgeon, is connected to the proximal end (13) of the barrel (11). The handle (14) is connected to a rod that extends along a central bore of the elongate barrel (11). The handle (14) can slide along a slot (18) formed in a proximal end portion (19) of the snare (10). Moving the handle (14) towards the proximal end of the snare (10) causes a loop of wire (17) to be retracted into the central bore of the elongate barrel (11). The distal end (12) of the snare consists of a flattened, widened portion (23) which has two cutting edges (21, 22). The cutting edges (21, 22) can be used in conjunction with the loop of wire (17) to assist in dissection of tissue. The snare may also include a vacuum port for removing smoke and blood from the site of the operation. The snare can be an eloctrosurgery snare.

### FIELD OF THE INVENTION

The present invention relates to a surgical snare that is suitable for use in removing protruding soft tissue, such as tonsils, baemorrhoids, polyps in the colon or the like.

### BACKGROUND OF THE INVENTION

Surgical snares have been in use for a considerable period of time. Conventional surgical snares operate by placing a wire loop around the tissue to be excised and retracting the wire loop into a tubular sheath or barrel. The wire loop cuts that tissue as it is retracted. Surgical snares may be constructed to allow the connection of an electrical source to the wire loop. When the wire loop is snug around the tissue to be excised, electrical current is delivered to the tissue in contact with the conductive wire loop. The electrical parent assists in cutting the tissue and cauterising the wound so-produced. Such snares may be monopolar or bipolar, as is commonly known in the art. Cutting or severing of tissue using electric current is frequently called diathermy.

Conventional surgical snares used in tonsillectomies comprise an elongate barrel of generally cylindrical construction having a central bore extending therethrough. The bore receives a holding means for holding the loop of wire. The holding means typically is a rod having one or two small holes located near the distal end thereof. The proximal end of the rod is typically connected to a handle that enables the surgeon to extend and retract the rod. When the rod is extended from the distal end of the barrel, the surgeon can insert respective ends of a wire into the holes to thereby form the wire loop. The rod is then retracted back into the barrel and the snare is ready for use in removing tissue.

One problem identified with such conventional snares relates to the figment and use of the wire. As the wire is to be used to cut or sever the tissue, it must be of relatively small diameter. This can lead to breakage during use, thereby necessitating replacement during the operative procedure. Other surgical snares are used in operative procedures to remove other soft tissue growths, such as polyps in the colon. These snares typically have the wire loop welded to the rod and the wire loop is arranged to conduct electric current. In addition to experiencing problems with breakage, the small diameter of the wire can limit the amount of electrical current that can flow through the wire when the snare is used in eloctrosurgery.

The palatine tonsils are a pair of masses of lymphoid tissue situated in the tonsillar floss in each lateral wall of the oropharynx. Their projecting remedial surfaces are each free and covered by a mucous membrane, while their lateral surfaces are each covered by a capsule that separates it from the superior constructor muscle of the pharynx. Removal of each palatine tonsil is relatively easy because of the rounded lateral aspect of the gland presented by the capsule. The tonsil is however loosely attached to underlying tissues and is richly supplied with blood vessels.

To remove a tonsil the mucous membrane covering the tonsil is typically cut and the tonsil loosened form the underlying tissue. A snare is then placed around the tonsil and drawn tight to cut the vessels and other tissues that are holding the tonsil to the underlying tissue. The blood vessels and connective tissue are ruptured by the snare is it is drawn tight.

Removal of haemorrhoids or polyps in the colon may take place in a similar fashion.

### SUMMARY OF THE INVENTION

It is an object of the present invention provide an improved surgical snare.

In a first aspect, the present invention provides a surgical snare characterised in that said snare has at least one cutting edge at a distal end thereof.

In a second aspect, the present invention provided a surgical snare for removing protruding soft tissue comprising an elongate barrel having a distal end and a proximal end, said barrel having a bore extending from the distal end towards the proximal end, said snare adapted to hold a loop of material such that said loop can be retracted into and extended from the bore of the barrel, wherein said snare has at least one cutting edge at or near said distal end.

Preferably, the distal end of the snare has an opening that is wider than a diameter or width of the bore of the barrel.

Preferably, the at least one cutting edge is formed in the material from which the snare is made. Alternatively, the at least one cutting edge may be formed by providing or mounting at least one cutting edge to the distal end of the snare. In this embodiment, the at least one cutting edge may be at least one scalpel blade or razor blade mounted to the distal end of the snare.

The distal end of the snare may be flattened so that two closely spaced edges are positioned relatively closely to each other. Especially preferably, two closely spaced edges are spaced just sufficiently to enable the loop to move through the opening substantially unhindered. In this embodiment, the snare preferably includes two cutting edges positioned on each of the flattened edges. The two cutting edges may be substantially identical distances from the proximal end of the snare. In another embodiment, one cutting edge may be positioned closer to the proximal end of the snare than the other cutting edge.

The loop may comprise a loop braided wire, or a loop of mono-filament wire. The wire may be an insulated wire or a non-insulated wire. The wire may be of larger diameter than is typically used in prior art snares. In prior art snares, the loop of material acts to cut the soft tissue. In contrast, in the present invention, the at least one cutting edge or the distal end of the snare cuts the larger proportion of the soft tissue, with the loop acting to ensnare the soft tissue and force it into contact with the cutting edge. In use of the snare of the present invention, the loop of material is passed or fitted around the protruding tissue and the surgeon begins to retract the loop onto the barrel of the snare. This brings the soft tissue into contact with the at least one cutting edge. Further retraction of the loop causes the soft tissue to be severed by the at least one cutting edge. Although not necessary for satisfactory operation of the present invention, the loop may also contribute to some cutting of the soft tissue.

Use of a larger diameter wire reduces the likelihood of breakage of the wire during the operative procedure, thus reducing the likelihood of having to change the wire during the operative procedure. This simplifies the operation and has the potential to reduce the required for the operation, as well as reducing the chance of injury to the operator in changing the sharp wire of earlier snares.

The loop may also take the form of a loop of a tape or other flat material, or indeed the loop may be made of material that can be used to bring the soft tissue into contact with the cutting edge.

It is also possible that one or more of the cutting edges may be electrically conductive to carry a current during the operation. The current may be fused to canteens the site of the operation. The cutting edge may be a maonopola electrode or part of a bipolar arrangement. In bipolar arrangements, the loop of material may comprise the other electrode, or a second cutting edge may form the other electrode. The electrical circuits and connections a otherwise conventional and need not be described further.

In embodiments where the distal end has an opening therein that is sized larger than the diameter or width of the bore, a further advantage of the present invention is that the loop can accommodate larger sized soft tissue protrusions than conventional snares. This is due to the fact that the loop, when being retracted, contacts the edges of the distal end of the snare and the edges of the distal end are spaced further apart at their widest position when compared to conventional snares.

The at least one cutting one cutting edge may be convexly shaped, have a concave shape or be a straight cutting edge. The shape of the cutting edge is not particularly critical to the operation and use of the present invention and the present invention encompasses all shapes of cutting edge.

In a further aspect, the present invention provides a surgical snare for removing protruding soft tissue characterised in that said snare has cutting means at a distal end thereof. The cutting means may be at least one cutting edge, a laser cutting means or an ultrasonic cutting means.

In another embodiment of the invention, the snare includes a vacuum port in fluid communication with the bore, said vacuum port being connectable to a source of vacuum which enables removal of blood and smoke from the distal of the snare via the bore. In this embodiment, the snare may further comprise loop actuating means for selectively extending and retracting the loop, said loop actuating means extending along the bore, In this fashion, any blockages that may occur in the bore, for example due to coagulated blood becoming blocked in the bore, may be dislodged by moving the actuating means.

In another embodiment, the barrel of the snare may have a curve therein at or near the distal end thereof. This may enhance the view of the sight of the operation for the surgeon. The curved barrel may also act as a retractor for moving partly severed tissue away from the distal end of the snare.

In a further embodiment, the snare may further comprise a deflector formed on or fitted to an outer surface of the snare, the deflector having an outer suffice for engaging tissue severed at the distal end of the snare to thereby push the severed tissue away from the snare. The deflector may have an obliquely extending outer surface that extends rearwardly and outwardly from the snare. Alternatively, the deflector may have an outer surface that can be moved from a first disposition having a relatively low profile to a second disposition having a relatively high profile. For example, the deflector may have one or more flexible portions positioned between its ends, with the one or more portions bowing or bending upwardly when the ends of the deflector are moved relatively together. In another embodiment, the deflector may include inflatable balloon means.

The deflector may be formed as part of the snare. Alternatively, the deflector may be formed as a separate item to the snare and fitted to the snare. In this embodiment, the deflector preferably includes fixing means for fixing the deflector to the snare. The fixing means may suitably comprise a snap fit fixing means for snap fitting the deflecting to the snare. Alternatively, the fixing means may comprise one or more loops that fit around the barrel of the snare. Other fixing means also fall within the scope of the present invention.

In embodiments where the snare is fitted with the vacuum port, the snare may further comprise one or more openings it a sidewall thereof, with the openings being positioned at or near the distal tip of the snare. The openings are in fluid communication with the vacuum port. In this fashion, the openings increase the area available for suction and removal of blood and smoke at the site of the operation. It may be appropriate in some instances to be able to control the area at the distal tip available for suction. To enable this, the snare may further comprise a blocking means for selectively blocking the one or more openings. For example, the blocking means may comprise a sleeve slidably positioned over the barrel of the snare, the sleeve being moveable from a closed position where it overlies the openings to an open position where it does not cover the openings.

In another aspect, the present invention provides a surgical snare characterised in that the snare has cutting at a distal end thereof.

The cutting means may comprise a cutting edge. The inventor has found that bleeding may be reduced if the cutting edge is relatively blunt.

Alteratively, the cutting means may be a diathermy cutting means, The diathermy cutting means may comprise an edge through which an electric current for severing tissue can pass. The snare may be a monopolar or bipolar snare in this embodiment.

In another embodiment, the cutting means in a sharp cutting edge and a diathermy current can also pass through the cutting means. The sharp cutting edge assists in keeping the severed tissue uniform and smooth whilst the diathermy current assists in cutting and cauterises at the same time. This allows the surgeon to avoid having to apply great pressure and use jerky cutting to sever the tissue. If jerky cutting is used, some of the severed tissue may miss out on uniform and proper coagulation.

The inventor has also found that the tissue being severed by the cutting means has a tendency to rest on the cutting means and the snare, thereby obscuring the operative site.

In a further aspect, the present invention provides a deflector for fitment to a surgical snare or a dissector, said deflector comprising fixing means fixing the deflector to the snare or dissector and an outer surface for engaging with tissue severed by a distal end of the snare or dissector to thereby push said tissue away from the snare or dissector.

In one embodiment, the deflector means has an obliquely-extending outer surface that extends rearwardly and outwardly from the snare or dissector when the deflector is fitted to the snare or dissector.

In another embodiment, the deflector means has an outer surface that can be moved from a first disposition having a relatively low profile to a second disposition having a relatively high profile. For example, the deflector means may have one or more flexible portions positioned between its ends, which one or more flexible portions bow or bend upwardly when it ends are moved relatively together. As the ends move relatively together, the upward bowing of one or more flexible portions gives the deflector means a relatively high profile. When in the first disposition of relatively low profile, the deflector means has its outer surface positioned relatively close to the outer surface of the snare or the dissector. When in the second disposition of relatively high profile, the deflector means has its outer surface positioned relatively far from the outer surface of the snare or dissector.

The outer surface of the deflector means may be continuous or discontinuous.

In another embodiment, the deflector means may include an inflatable balloon means.

The fixing means may comprise a snap-fit fixing means for snap fitting the deflector means to the snare. Alternatively, the fitting means may comprise one or more loops that fit around the barrel of the snare or dissector. It will be apparent to the person skilled in the art that a number of other fixing means may also be used.

The deflector means may be manufactured as a separate item and subsequently fitted to the snare or dissector. Alternatively, the deflector means may be integrally formed with the snare or dissector.

In yet a further aspect, the present invention provides a surgical snare or dissector having a barrel, cutting means at a distal end of the barrel and deflector means on said barrel, said deflector means adapted to engage with tissue that has been severed by the cutting means to thereby push said severed tissue away from the snare or dissector.

The part of tie deflector means that engages with the tissue may be as described with respect to the second aspect of the present invention.

Surgical snares include a barrel portion having a central bore extending therethrough. This central bore provides a passageway through which the loop of material or a control rod holding the loop of material passes. The loop of material or control rod can be moved forwardly and rearwardly along this bore to respectively extend and tract the loop. When the snare is used to sever tissue, bleeding occurs and the blood can obscure the site of the operation. Furthermore, if the snare is an electrosurgical snare, the electric current can cause smoke to be generated. In conducting some procedures, it is necessary for the surgeon to place his head in close proximity to the site of the operation. This can cause the surgeon to come into close contact with the smoke, which can be quite unpleasant.

In a still further aspect, the present invention provides a surgical snare having a barrel, a bore in the barrel, a loop of material extending from a distal end of the bore, loop actuating means for selectively extending and retracting the loop and connection means connecting the loop of material to the loop actuating means, characterised in that said snare further comprises a vacuum port in fluid communication with the bore, said vacuum port being connectable to a source of vacuum.

In this aspect, a suction line can be connected to the vacuum port. This enables blood and smoke to be removed from the site of the operation via the bore of the snare when vacuum is applied to the vacuum port via the suction line. If any blockages occur in the bore, movement of the connection means along the bore can assist in clearing such blockages.

The connection means typically comprises a rod having attachment means in a distal end for attaching the loop of material thereto. The proximal end of the rod is connected to the loop actuating means. The loop actuation means usually comprises a gripping means that can be moved forwardly and rearwardly along the longitudinal direction of the snare.

It is preferred that a reduced or minimum gap be present between the connection means and the proximal end of the bore in order to reduce air leakage at the proximal end of the bore. It will be appreciated that the bore has one or more large openings at the distal end thereof and thus a degree of air leakage at the proximal end of the bore can be tolerated whilst still maintaining adequate removal of blood and/or smoke at the distal ends.

In this embodiment, the snare may also be provided with one or more openings in a side wall thereof, said openings being positioned at or near the distal tip of the snare, said also being in fluid communication with the vacuum part. Preferably, the one or more openings in the sidewall are in fluid communication with the bore.

The one or more openings provide further openings of removing blood and smoke from the site of the operation.

The snare may also be provided with a sleeve slidably positioned over the barrel of the snare, said sleeve being movable from a closed position where it overlies said openings in the sidewall thereof to an open position where it does not cover the openings. In this fashion, the area available for suction at the distal tip of the snare can be varied.

The provision of vacuum through the bore of the snare simplifies construction of the snare in that a separate vacuum bore does not need to be provided. Furthermore, the connection means may be moved inwardly and outwardly to assist in removing any blockages that may occur.

The present inventor has also found that providing the snare with a curve at or near the distal end thereof can improve vision of the operative site.

According to yet a further aspect, the present invention provides a surgical snare characterised in that it has at least one curve at or near the distal end thereof.

It has been found that the curve in the snare not only improves vision of the operative site but also acts as a retractor.

The present invention has also proposed a system for eloctrosurgery in which electrical current is provided to the loop of material that extends from the distal end of the snare and then passes to the barrel of the snare.

According to another embodiment, the present invention provides a surgical snare having an electrically conductive barrel and a loop of electrically conductive material extending out from a bore in the barrel, wherein electric current is supplied the loop of material and the electric current feeds into the barrel of the snare. Preferably, the barrel is made from electrically conductive material and all but a tip of the barrel is covered with an electrically insulating material. Preferably, the loop of material contacts the barrel, most preferably the tip of the barrel, to thereby cause electric current to flow into the barrel. The loop may be provided with electrical contact means to enable an electric current be fed thereto. Preferably, the electrical contact means feed electric current to the rod holding the loop.

The snare of this aspect of the invention may be a monopolar electrosurgical device or a bipolar electrosurgical device.

In an alternative embodiment of this aspect of the present invention, electric current is supplied to the barrel and feeds into the loop of material. In this embodiment, the barrel may be provided with electric contact means to enable an electric current to be fed thereto.

The various features of each of the aspects of the present invention may be used separately or in combination with one or more features of the other aspects of the present invention.

### BRIEF DESCRIPTION OF THE DRAWINGS

The present invention will now be further described with reference to preferred embodiments thereof as shown in the accompanying drawings. In the drawings:

FIG. 1 shows a plan view of a surgical snare in accordance with the present invention;

FIG. 2 shows a plan view of the distal end of the surgical snare of FIG. 1;

FIG. 3 shows a side view of the distal end of the surgical snare of FIG. 1;

FIG. 4 shows an underneath view of the distal end of the surgical snare in accordance with another embodiment of the present invention;

FIG. 5 shows a side view of the distal end of the surgical snare shown in FIG. 4;

FIGS. 6 to 9 show the snare of FIGS. 1 to 3 being used to sever protruding soft tissue;

FIGS. 10 and 11 show two ways of mounting the loop of wire to a rod that extends along a central bore of the barrel of the snare;

FIG. 12 shows an embodiment of a snare in accordance with the present invention having a concave cutting edge;

FIG. 13 shows a top view of a snare having a deflector in accordance with the present invention;

FIG. 14 shows a side view of the snare of FIG. 13;

FIG. 15 shows a top view of the snare of FIG. 14 with the loop extended;

FIG. 16 shows an alternative embodiment of a snare with deflector in accordance with the present invention;

FIG. 17 shows a top view of a snare having a vacuum port in accordance with another aspect of the present invention;

FIG. 18 shows a plan cross-sectional view of part of the snare of FIG. 17;

FIG. 19 shows a side cross-sectional view of the snare of FIG. 17;

FIG. 20 shows a side view of part of the snare of FIG. 17 with a vacuum line attached thereto; and

FIG. 21 shows a side view of a snare in accordance with another embodiment of the present invitation.

It is to be understood that the attached Figures show preferred embodiments of the present invention and that the present invention should not be limited to the features shown in the Figures.

### DETAILED DESCRIPTION OF THE EMBODIMENTS

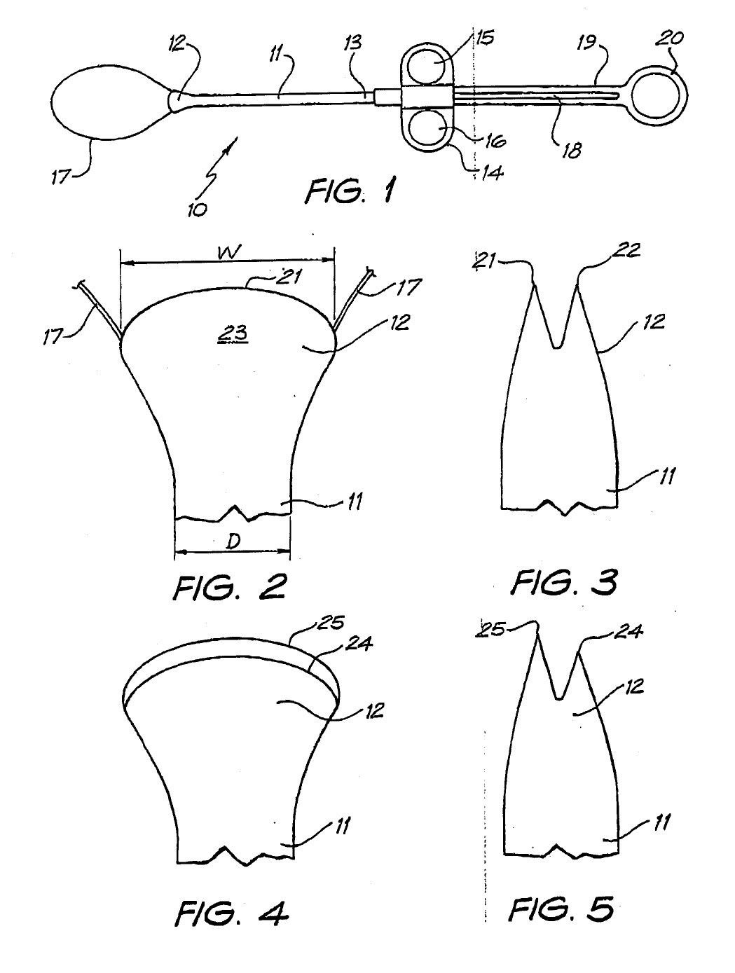

The surgical snare 10 shown in FIG. 1 comprises an elongate barrel 11 having a distal end 12 and a proximal end 13. A handle 14 having openings 15, 16 which can accommodate the fingers of the surgeon, is connected to the proximal end 13 of the barrel 11. The handle 14 is connected to a rod (not shown) that extends along a central bore of elongate barrel 11. The rod (not shown) includes mounting means for mounting a loop of wire 17 thereto. The handle 14 can slide along a slot 18 formed in a proximal end portion 19 of the snare 10. Moving the handle 14 towards the proximal end 20 of snare 10 causes the loop of wire 17 to be retracted into the central bore of the, elongate barrel 11.

Turning now to FIGS. 2 and 3, the distal end 12 of barrel 11 includes two cutting edges 21, 22. As best shown in FIG. 2, the elongate barrel 11 has A diameter D. The distal end 12 of the snare consists of a flattened, widened portion 23 that has a width W. As best shown in FIG. 2, width W is somewhat larger than diameter D. As a result of the width W being larger than diameter D, the loop of wire 17 can extend back into the central bore of barrel 11 at the widest point of the flatted portion 23 of distal end 12, thereby resulting in the loop 17 having a larger dimension at the point where the wire enters the distal end of the snare 10, where compared with convention snares that have a straight distal end. This shows the loop of wire 17 of the snare 10 of the present invention to entrap or ensnare larger masses of protruding soft tissue. In the embodiments shown in FIGS. 2 and 3, the cutting edges 21 and 22 provided by shaping the distal end 12 of snare 10 to form the sharp cutting edges. In other embodiments, the cutting edges may be provided by mounting one or more blades to the distal end 12 of the snare 10.

As can be seen from FIGS. 2 and 3, the cutting edges 21, 22 are positioned at essentially the same distance from the proximal end 20 of snare 10. In other embodiments of the present invention, as best shown in FIGS. 4 and 5, cutting edge 24 may be located at a different distance from the proximal end 20 of snare 10 that cutting edge 25. In particular, as shown in FIG. 4, cutting edge 24 is positioned slightly offset in a longitudinal direction to cutting edge 25.

Use of the snare to remove protruding soft tissue is shown in FIGS. 6 to 9. In FIG. 6, the snare 10 is positioned such that the loop of wire 17 ensnares the protruding soft tissue 26 to be removed. The loop of wire 17 is then retracted into snare 10 by mutation of handle 14. This brings the cutting blades 21, 22 into contact with the soft tissue 26 and cutting blades 21, 22 commence cutting the soft tissue 26. This is best shown in FIG. 7.

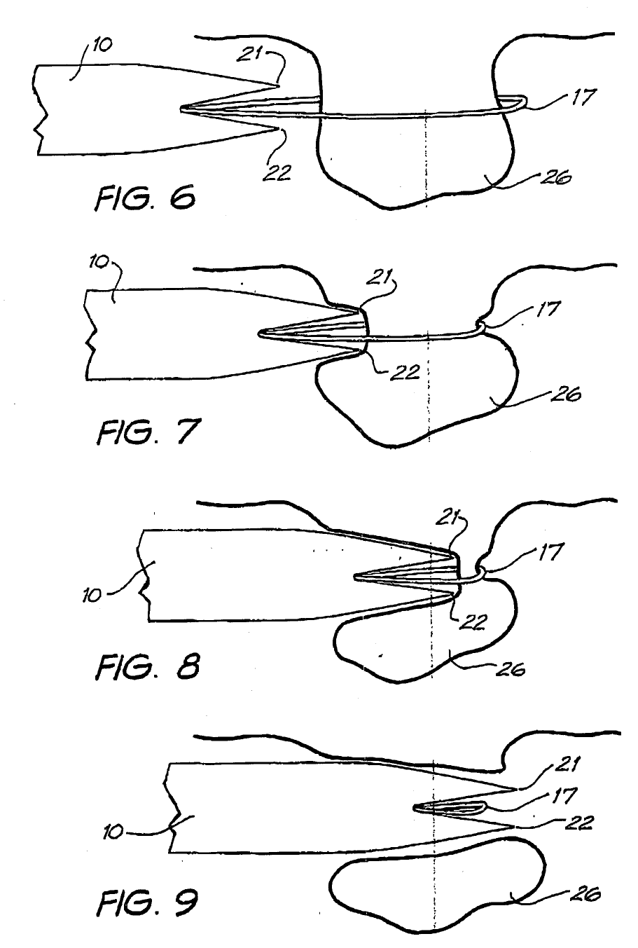

Continued retraction of the loop of wire 17, as best shown in FIG. 8, results in the cutting edges 21, 22 further severing the mass of tissue 26 until the mass of tissue 26 has been completely severed as shown in FIG. 9. In FIG. 9, the loop of wire 17 is essentially retracted into the snare 10.

The surgical snare shown in the embodiment of FIGS. 1 to 5 is preferably made from a rigid plastics material such that it may be disposable. The loop of wire 17 is partly a braided wire or a multi-filament wire. Such wires are less prone to breakage.

It is also possible that the cutting edges 21 and 22 as shown in FIGS. 1 to 3, or 24 and 25, as shown in FIGS. 4 and 5, may be made from an electrically conductive material and the cutting edges may be the electrodes of an electro-surgical apparatus that is operable such that an electric current flows through the blades and acts to cauterise the soft tissue 26 that is being excised or removed in the operation.

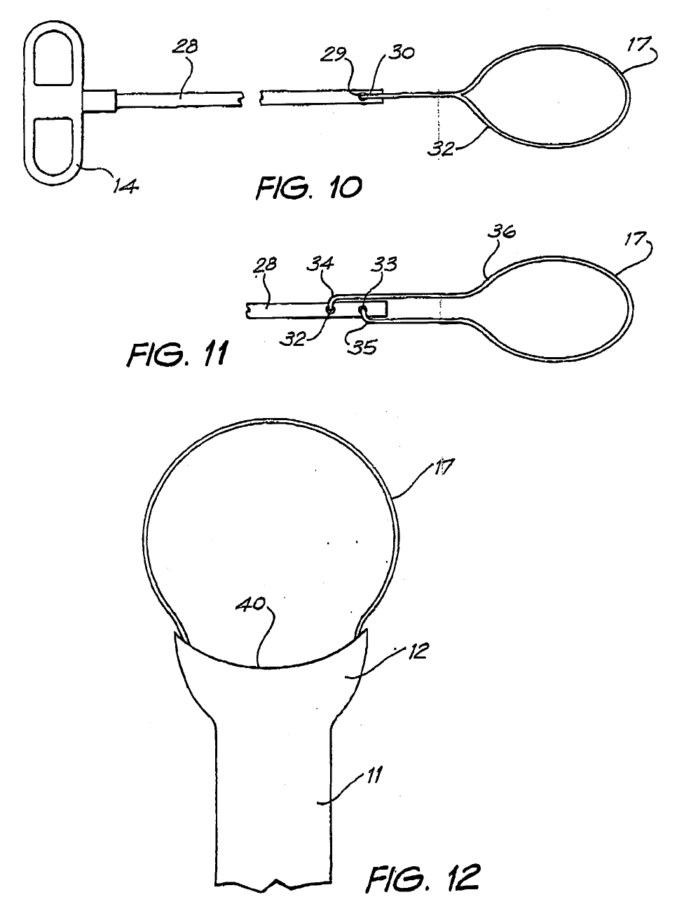

FIG. 10 shows one embodiment for mounting the loop of wire 17 to a rod 28 that is connected to handle 14, which rod 28 extends through the central bore of elongate barrel 11. In particular, rod 28 may be provided with a hole 29 near its distal end, which hole 29 is adapted to receive and hold an end 30 of a piece of wire 31 having a loop 32 formed or attached to its end. In this embodiment, it is only necessary to insert the end 30 of wire 31 into hole 29 to affix the loop of wire 17 to the rod 28.

The embodiment of FIG. 11 shows a further way of mounting the loop of wire 17 to rod 28. In particular, rod 28 may be provided with two holes 32, 33 adapted to receive respective ends, 34, 35 of a piece of wire 36 to thereby form loop of wire 17.

Turning now to FIG. 12, which shows a plan view of the end portion of a snare in accordance with another embodiment of the present invention, it will be seen that FIG. 12 is generally similar to FIG. 2, with the exception that cutting edge 40 shown in FIG. 12 is a concave cutting edge. The other features of FIG. 12 that are similar to features shown in FIG. 12 are identified by the same reference numeral as even in FIG. 2 and not be described further.

FIGS. 13 to 15 show a surgical snare having a deflector means in accordance with the present invention fit thereto. As shown in FIGS. 13 to 15, the snare 110 has an elongated barrel 11 and a handle portion 112. The distal end 113 of the snare 110 includes a cutting edge 114. A loop of material 115 (best shown in the extended position in FIG. 15) extends from a bore (not shown) in the barrel 111 of snare 110. Loop of material 115 can be selectively extended and retracted by manipulation of suitable actuating means (not shown) located on handle portion 112.

The snare 110 is fitted with deflector mean 116 for moving severed tissue away from the distal end 113 of the snare during a surgical operation utilizing the snare. The deflector means 116 includes a distal end 117 having a loop 118 that passes around the outside of barrel 111 of snare 110. The deflector means also includes a proximal end 119 having a loop 120 that also extends around the external diameter of barrel 11. Proximal end 19 of deflector means 116 further includes a thumb or finger engaging portion 121 that facilitates movement of the proximal end 119 relatively closer to the distal end 117.

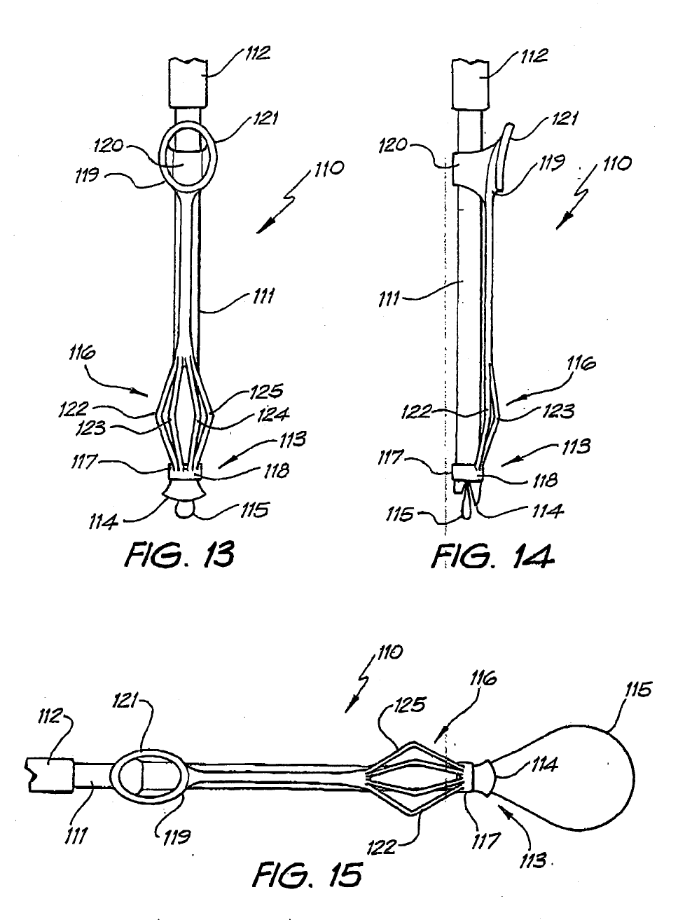

The deflector means 116 further includes a plurality of flexible members 122, 123, 124, 125. As can best be seen from FIG. 14, flexible members 122 to 125 include intermediate portions that are spaced from the barrel 111 of the snare. Thus, the flexible members 122 to 125 can act to deflect severed tissue away from the snare. If further space is required between the severed tissue and the snare, thumb or finger engaging portion 121 can be used to push the proximal end 119 of the deflector means towards the distal end 117 of the deflector means. This causes the flexible members 122 to 125 to bow upwardly such that their intermediate portions are spaced a greater distance from the barrel 111 of snare 110. This is best shown in FIG. 15.

FIG. 16 shows an alternative embodiment to that shown in FIGS. 13 to 15. In FIG. 16, the snare 110 which has a barrel 111 and a handle portion 112 is generally identical to the snare 110 shown in FIGS. 13 to 15. Similarly, the snare 110 of FIG. 16 includes a cutting edge 114 located at a distal end 113 thereof. A loop of material 115, such as a loop of wire, extends from a bore (not shown) in the barrel of the snare. FIG. 16 differs from the embodiment shown in FIGS. 13 to 15 in that it includes a deflector means 130 having a first loop 131 and a second loop 132 that pass around and engage the outer surface of barrel 111. This acts to hold the deflector means 130 in place. The deflector means 130 includes an obliquely extending outer surface 133 that extends rearwardly and upwardly from distal end 131 of the deflector means (and from distal end 113 of snare 110).

Deflector means 130 may suitably be made from a relatively rigid plastics material.

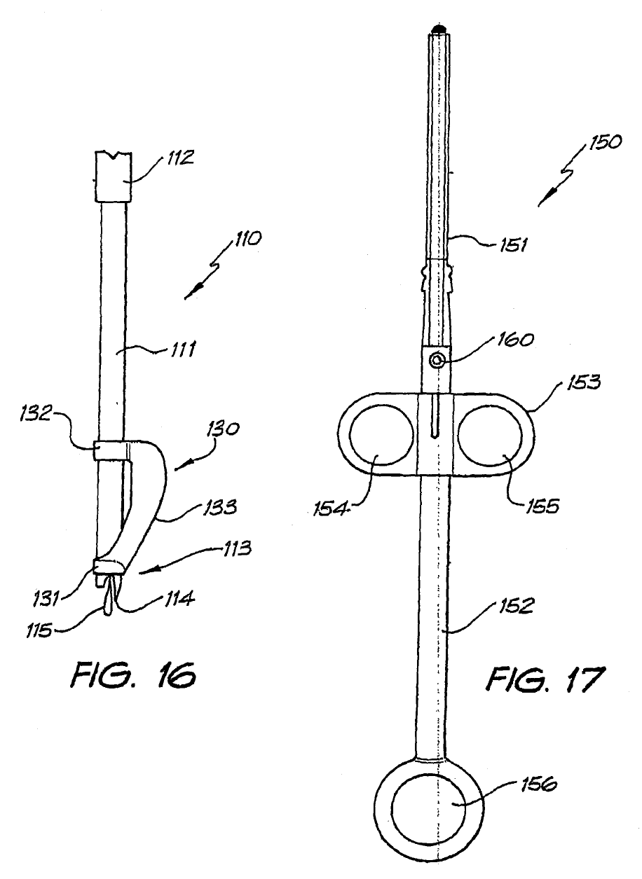

FIG. 17 shows a top view of a surgical snare in accordance with another embodiment of the present invention. The snare 150 has a barrel portion 151 and a handle portion 152. Handle portion 152 has an actuating means 153 solidly mounted thereto, Actuating means 153 includes two holes 154, 155 through which two fingers can be inserted. The end of the handle 152 has a further hole 156 formed thereon and this hole is adapted to receive the thumb of the user, Inserting fingers through holes 154, 155 and thumb though hole 156 enables the actuating means 153 to be easily manipulated by the surgeon.

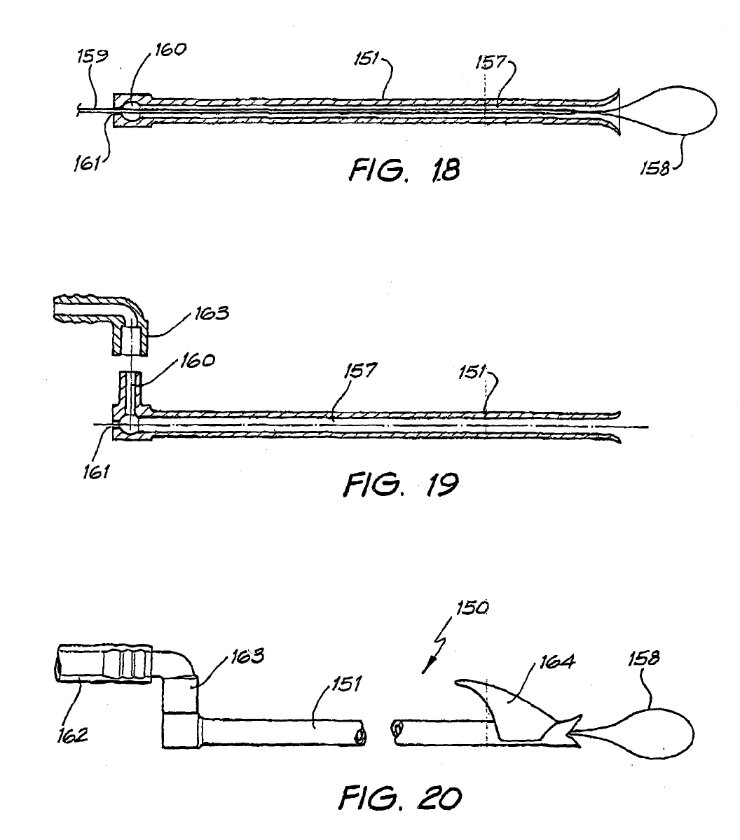

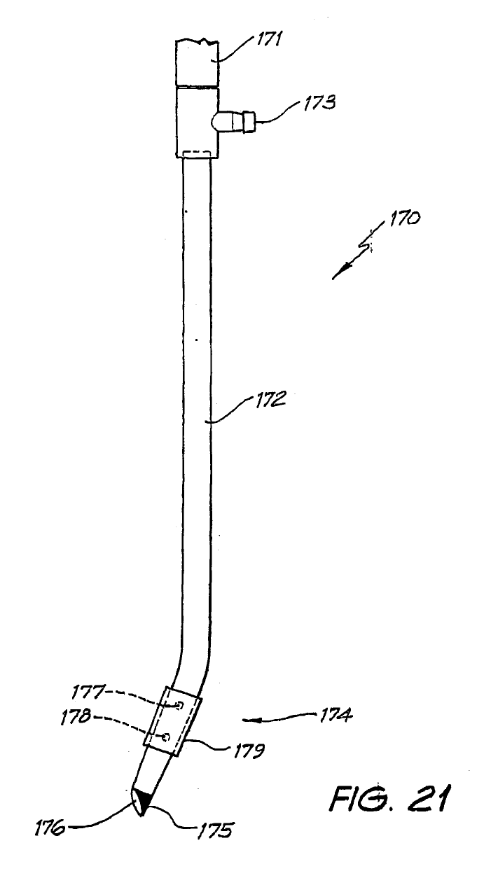

The barrel 151 has a bore 157 extending therethrough. The bore 157 is best shown in FIGS. 18 and 19. A loop of material 158 extends from the distal end of the ban 157. Loop of material 158 is connected to a rod 159, which rod is itself connected to the actuating means 153. Moving the actuating means 153 in a longitudinal direction can cause extension and reaction of the loop of material 158.

The snare 150 further includes a vacuum port 160. As rest shown in FIG. 19, vacuum port 160 is in fluid communication with bore 157. As will a be noticed from FIGS. 18 and 119, the proximal end of the bore 157 has an opening 161 of reduced diameter such that the walls of opening 161 come into contact with the outer diameter of rod 159. This assists in achieving an essentially air tight seal around rod 159 such that air leaks between the walls of the bore and the rod are reduced when vacuum is applied to the vacuum port 160.

In order to facilitate attachment of a suction line 162 to vacuum port 160, an adapter piece 163 may be provided. Alternatively, suction line 162 may be connected directly to vacuum port 160.

It will be seen from FIG. 20 that the snare 150 is also provided with a releaseable detachable deflector means 164. Deflector means 164 includes an obliquely extending outer surface that moves upwardly and rearwardly from the distal end of the snare 150.

FIG. 21 shows a further embodiment of the present invention. In particular FIG. 21 shows a surgical snare 170 having a handle 171 and a barrel 172. A vacuum port 173 is provided at the proximal end of the barrel 172.

As can be seen from FIG. 21, the barrel 172 includes a curved tip portion 174. The curved tip portion gives better vision to see the site of the operation. The curved tip also works as a retractor in that it can be used to move severed tissue away from the tip of the snare.

The distal end of the snare includes a diathermy dissector 175. In this regard, it is preferred that the barrel 172 of snare 170 is made from a metallic or electrically conductive material such that electric current can flow along the barrel. It is preferred that the bulk of the length of the barrel is covered with an electrically insulating material such that current cannot flow from the barrel to the patient. However, the tip of the barrel is preferably left uncovered such that electric current can flow from the tip into the tissue of the patient to thereby sever the tissue by diathermy.

The tip of the barrel includes an opening 176 which is an opening for suction applied through vacuum port 173 as well as an opening through which the loop of material also extends.

In the embodiment shown in FIGS. 17 to 21, the bore in the barrel of the snare provides a conduit for suction applied to the site of the operation. In this way, suction can be applied to remove blood and smoke from the site of the operation without having to provide any extra conduits in the barrel of the snare. Moreover, the bore also provides the conduit through which the rod that connects the loop of material to the actuating means travels. If there are any blockages caused in this barrel, for example, by bringing clotted blood into the barrel, movement of the rod up and down in the barrel can assist in clearing those blockages.

The snare may be provided with one or more openings 177, 178 in the sidewall of the barrel of the snare and located at or near the distal tip of the snare. A sleeve 179 may be slidably mounted over the barrel to selectively cover and uncover the openings 177, 178 to control the area available for suction at or near the distal tip of the snare. Openings 177, 178 are suitable in fluid contact with the bore of the snare.

In embodiments of the present invention that include a vacuum port, the snare has particular advantages that allow reduced operating time during tonsillectomy procedures. In particular, the snare of this embodiment allows the surgeon to continually suck smoke and blood away from the site of the operation. This enables the surgeon to clearly see the origin of bleeding. As a result, the bleeding can be rapidly cauterized (in preferred embodiments of the invention, by using the tip or the loop of the snare). This reduces the amount of blood loss to a minimum.

Furthermore, there is no need for an assistant or nurse to place his or her head in the field of view and use a separate sucker to remove blood and smoke. Using conventional instruments can result in blood and smoke collecting in the cavity, This can increase the total loss of blood and increase the the required to complete the procedure. This is undesirable as it increases theatre time (thereby reducing the usage of the theatre), increases the amount of an anaesthetic used and increases the time that the patient is under the anaesthetic. These disadvantages can be ameliorated by at least preferred embodiments of the present invention.

The snare of some preferred embodiments of the present invention can also reduce the equipment requirements for operations in that:

a) there may be no need for a separate diathermy pencil; and

b) there may be no need for a separate sucker during tonsil dissection.

Some of the above benefits may also be realised if the snare is used in other operations. It will be appreciated that the snare of the present invention is primarily for use in tonsillectomies. However, it will also be understood that the snare is not necessarily limited to that use and it may be used in other operations.

It should also be noted that the tip of the snare or barrel can also be used as a dissector.

It will be understood that the invention disclosed and defined herein extends to all alternative combinations of two or more of the individual features mentioned or evident from the text or drawings. All of these different combinations constitute various alternative aspects of the invention.

The foregoing describes embodiments of the present invention and modifications, obvious to those skilled in the art can be made thereto, without departing from the scope of the present invention.

1. A surgical snare for removing protruding soft tissue comprising an elongate barrel having a distal end and a proximal end, said barrel having a bore extending from the distal end towards the proximal end, said snare adapted to hold a loop of material such that said loop can be retracted into and extended from the bore of the barrel, wherein said snare has at least one cutting edge at or near said distal end.

2. A snare according to claim 1 wherein the distal end of the snare has an opening that is wider than a diameter or width of the bore of the barrel.

3. A snare as claimed in claim 2 wherein the distal end of the snare is flattened so that two closely spaced edges are positioned relatively closely to each other.

4. A snare as claimed in claim 3 wherein the two closely spaced edges are spaced just sufficiently to enable the loop to move through the opening substantially unhindered.

5. A snare as claimed in claim 2 wherein the snare has a cutting edge positioned on each of the closely spaced edges and the two cutting edges are positioned at substantially identical distances from the proximal end of the snare.

6. A snare as claimed in claim 2 wherein the snare is a cutting edge positioned on each of the closely spaced edges and one cutting edge is positioned closer to the proximal end that the other cutting edge.

7. A snare as claimed in any one of the preceding claims wherein the cutting edge comprises a diathermy cutting means comprising an edge through which an electric current for severing tissue can pass.

8. A snare as claimed in claim 7 wherein the snare is a monopolar snare or a dipolar snare.

9. A snare as claimed in any one of the preceding claims further including a vacuum port in fluid communication wit the bore, said vacuum port being connectable to a source of vacuum to enable removal of blood and smoke from distal end via the bore.

10. A snare as claimed in claim 9 further comprising loop actuating means for selectively extending and retracting the loop, said loop actuating means extending along the bore.

11. A snare as claimed in any one of the preceding claims wherein the barrel bas a curve therein at or near the distal and thereof.

12. A snare as claimed in any one of the claims 1 to 11 further comprising a deflector formed on or fitted to an outer surface of the snare, said defector having an outer surface for engaging tissue severed at the distal end of the snare to thereby push said tissue away from the snare.

13. A snare as claimed in claim 12 wherein the deflector has an obliquely extending outer surface that extends rearwardly and outwardly from the snare.

14. A snare as claimed in claim 12 wherein the deflector has an outer surface that can be moved from a first disposition having a relatively low profile to a second disposition having a relatively high profile.

15. A snare as claimed in claim 14 wherein the deflector has one or more flexible portions positioned between its ends, said one or more positions bowing or bending upwards when said ends are moved relatively together.

16. A snare as claimed in claim 14 wherein said deflector include inflatable balloon means.

17. A snare as claimed in any one of claims 12 to 16 wherein the deflector includes fixing means for fixing to the snare.

18. A snare as claimed in clause 17 wherein the fixing means comprises a snap-fit fixing means for snap fitting the deflector to the snare.

19. A snare as claimed in claim 17 wherein the fixing means comprises one or more loops that fit around the barrel of the snare.

20. A snare as claimed in any one of the claims 12 to 16 wherein the deflector is formed integrally with the snare.

21. A snare as claimed in claim 9 or claim 10 further comprising one or more openings in a side wall thereof, said openings being positioned at or near the distal tip of the snare, said opening being in fluid communication with the vacuum port.

22. A snare as claimed in claim 21 wherein the one or more openings are a fluid communication with the bore.

23. A snare as claimed in claim 21 or claim 22 further comprising blocking means for selectively blocking sad one or more openings.

24. A snare as claimed in claim 23 wherein said blocking means comprises a sleeve slidably positioned over the barrel of the snare, said snare being movable from a closed position where it overlies said openings to an open position where it does not cover said openings.
